(19) United States
(12) Patent Application Publication
(10) Pub. No.: US 2012/0214870 A1
(43) Pub. Date: Aug. 23, 2012

(54) METHODS FOR DIAGNOSIS AND TREATMENT OF CHRONIC FATIGUE SYNDROME

(75) Inventors: Allan Mark Evans, Rosslyn Park (AU); Stephanie Elizabeth Reuter, Marden (AU); Peter Lance Wigley, Keswick (AU)

(73) Assignee: UNIVERSITY OF SOUTH AUSTRALIA, Adelaide (AU)

(21) Appl. No.: 13/391,443

(22) PCT Filed: Aug. 30, 2010
(86) PCT No.: PCT/AU10/01115

§ 371 Date: May 4, 2012

(30) Foreign Application Priority Data
Aug. 31, 2009 (AU) 2009904136

(51) Int. Cl. A61K 31/205; A61P 43/00; A61K 31/203; G01N 33/53

(52) U.S. Cl. 514/556; 436/501; 514/560

(57) ABSTRACT

Methods for the diagnosis and treatment of chronic fatigue syndrome (CFS) are disclosed based upon the finding that particular individual acylcarnitines are present in modified concentrations (ie decreased or increased concentrations) in CFS patients compared to healthy control subjects. In one form of the invention, a diagnostic method comprises determining a concentration of at least one individual acylcarnitine compound (eg oleyl-L-carnitine and linoleyl-L-carnitine) in a body sample from a test subject and comparing the concentration to a reference concentration, wherein a difference in the concentration of the at least one individual acylcarnitine from the test subject compared to the reference concentration is indicative of CFS. In another form of the invention, a method of treating CFS is provided which comprises administering an effective amount of a supplement comprising: at least one acylcarnitine compound selected from short-chain, medium-chain and long-chain acylcarnitines, L-carnitine (or an acylcarnitine that may be converted within a subject to L-carnitine) in combination with at least one fatty acid selected from short-chain, medium-chain and long-chain fatty acids, or at least one acylcarnitine in combination with at least one fatty acid selected from short-chain, medium-chain and long-chain fatty acids.

### FIELD OF THE INVENTION

The present invention relates to methods for the diagnosis and treatment of chronic fatigue syndrome.

### BACKGROUND OF THE INVENTION

Chronic fatigue syndrome (CFS), also known as Myalgic Encephalomyelitis (ME), is a term used to describe a heterogeneous, multi-systemic condition which is primarily characterised by persistent debilitating fatigue that cannot be attributed to any alternative condition. The underlying aetiology of CFS is unknown and no diagnostic test presently exists. Instead, CFS is presently diagnosed on subjective symptomology, wherein other medical conditions that may explain the symptoms have been ruled out. Specifically, the subject must have clinically-evaluated, unexplained, persistent or relapsing fatigue for six months or more, that: (1) is of new or definite onset; (2) is not the result of ongoing exertion; (3) is not substantially alleviated by rest; and (4) results in a substantial reduction in previous levels of occupational, educational, social or personal activities. Additionally, the subject must have four or more of the following symptoms that are concurrent, persistent for six months or more and which do not predate the fatigue: (1) impaired short-term memory or concentration; (2) sore throat; (3) tender cervical or axillary lymph nodes; (4) muscle pain; (5) multi-joint pain without arthritis; (6) headaches of a new type, pattern, or severity; (7) unrefreshing sleep; and (8) post-exertional malaise lasting more than 24 hours (Royal Australasian College of Physicians Working Group, 2002). A variety of biochemical factors have been associated with CFS, including depressed mitochondrial respiration and alteration in carnitine homeostasis; however, no causative links have presently been established.

Carnitine is an important endogenous compound that is found in all mammalian species (Bremer, 1983), with L-carnitine being the biologically active form of carnitine. Generally, adequate levels of L-carnitine are obtained from dietary sources, particularly from red meat, and L-carnitine is additionally biosynthesised in the kidneys, liver and to some extent in the brain (Bremer, 1983). However, alterations in carnitine homeostasis can have a detrimental effect on human health. For example, in its severest form, carnitine deficiency is associated with progressive cardiomyopathy, encephalopathy and muscle weakness, resulting in death from heart failure (Pons and de Vivo, 1995; Scholte et al. 1990).

Carnitine transports long-chain acyl groups of fatty acids across the inner mitochondrial membrane which is important for energy production in a process known as fatty acid β-oxidation, wherein fatty acids are metabolised to produce energy. As depicted in FIG. 1, the acyl group of a fatty acid is transferred to Coenzyme A (CoA), an acyl group carrier. Next, an enzyme known as carnitine palmitoyltransferase (CPT)-I (also known as carnitine acyltransferase-I) catalyses the transfer of the acyl group from CoA to L-carnitine in a reaction referred to as an “acyltransferase reaction” and the resulting acylcarnitine is capable of crossing the inner mitochondrial membrane via a L-carnitine/acylcarnitine translocase. Once across the inner membrane of the mitochondria, the acyl group is transferred from the L-carnitine molecule to a mitochondrial CoA, a reaction known as a “reverse transesterification” or “reverse acyltransferase reaction” catalysed by carnitine palmitoyltransferase (CPT)-II (also known as carnitine acyltransferase-II). The resulting acylCoA molecule then enters the fatty acid β-oxidation pathway where it is broken down to produce energy via the Krebs cycle (also known as the citric acid cycle and the tricarboxylic acid (TCA) cycle). The particular individual acylcarnitine that is formed during this process is dependent upon the particular individual fatty acid, or more precisely, the particular individual alkyl group (ie the aliphatic hydrocarbon chain component) of the acyl group of the fatty acid. This alkyl group may be of a variable length (eg 4 to 32, or more, carbon atoms); be saturated, mono-unsaturated or poly-unsaturated; be linear or branched; and be hydroxylated or contain a carboxylic acid moiety.

It has been suggested that due to the important role of L-carnitine in fatty acid oxidation and accordingly, energy metabolism, alterations in L-carnitine and acylcarnitine homeostasis may be associated with fatigue observed in CFS (Kuratsune et al., 1997). In this regard, some studies have reported a reduction in endogenous plasma L-carnitine and total carnitine concentrations (Kuratsune et al., 1998; Plioplys and Plioplys, 1995). However, conflicting results have been obtained in other studies (Kuratsune et al., 1994; Kuratsune et al., 1995; Jones et al., 2005; Majeed et al., 1995). Similarly, CFS has been associated with reduced levels of endogenous total acylcarnitine levels in some studies (Kuratsune et al., 1994; Kuratsune et al., 1995; Kuratsune et al., 1998), whilst other studies have reported no difference between such levels in CFS patients and healthy controls (Jones et al., 2005; Majeed et al., 1995). Notably, these studies have not examined levels of individual acylcarnitines, but rather total acylcarnitine levels (ie the sum of all individual acylcarnitines). Consequently, alterations in the levels of a particular individual acylcarnitine in these patients may be masked by relatively normal levels of other individual acylcarnitines.

Another study found that acylcarnitine was not decreased in CFS patients, and further that there were no significant difference in the level of total carnitine, free carnitine and 20 different acylcarnitine compounds between CFS patients and healthy controls (Soetekouw et al., 2000). However, this study utilised a lower quantification limit that was well above the levels reported for all of the medium- and long-chain acylcarnitine compounds quantified, and only about two-thirds of the individual acylcarnitines were analysed; accordingly, it is not possible to draw firm conclusions regarding the carnitine pool composition in CFS patients from that study.

L-carnitine supplementation has been shown to significantly reduce fatigue severity in CFS patients after 2 months of supplementation (Plioplys and Plioplys, 1997). Similarly, supplementation with acetyl-L-carnitine (ALC) has been observed to result in significant improvements in mental fatigue and attention concentration (Vermeulen & Scholte; 2004; Malaguarnera et al. 2008). Further, administration of propionyl-L-carnitine (PLC) resulted in significant improvements in general and physical fatigue (Vermeulen & Scholte, 2004). Previous studies have also demonstrated that administration of essential fatty acids results in a significant improvement in CFS symptomology (Puri, 2004; Puri, 2007; Tamzi far and Tamzi, 2005). However, the exact deficiencies in CFS are not well defined, and accordingly, none of these supplements specifically target deficiencies in CFS.

The present applicant has now found that the concentration of a number of individual acylcarnitines is decreased in CFS, and that others are present at an increased concentration in CFS, compared to healthy controls. Further, it has been realised that the individual acylcarnitines that are present at a modified concentration may be utilised to diagnose CFS. Moreover, it has been realised that this finding enables the rational design of novel methods for treatment of CFS.

### SUMMARY OF THE INVENTION

Thus, in a first aspect, the present invention provides a method of diagnosing chronic fatigue syndrome (CFS) in a test subject, said method comprising the steps of:
(i) determining a concentration of at least one individual acylcarnitine compound in a body sample from the test subject, and
(ii) comparing the concentration determined in step (i) to a reference concentration of the at least one individual acylcarnitine determined from an equivalent body sample from a healthy control subject (or a reference concentration range of the at least one individual acylcarnitine determined from equivalent body samples from a plurality of healthy control subjects),
   wherein a difference in the concentration of the at least one individual acylcarnitine from the test subject compared to the reference concentration (or reference concentration range) is indicative of CFS in the test subject.

In a second aspect, the present invention provides a method of diagnosing chronic fatigue syndrome (CFS) in a test subject, said method comprising the steps of:
(i) determining a concentration of at least one individual acylcarnitine compound in a first body sample from the test subject;
(ii) determining a concentration of at least one individual fatty acid that corresponds to an acyl group of said at least one individual acylcarnitine compound in a second body sample from the test subject, wherein said first and second body samples may be the same; and
(iii) determining a ratio of the concentration of the at least one individual acylcarnitine compound to the concentration of the at least one individual fatty acid, or
   assessing a relationship between the concentration of the at least one individual
acylcarnitine compound and the concentration of the at least one individual fatty acid;
   wherein an aberrant ratio determined in step (iii) or an aberrant relationship assessed in step (iii) is indicative of CFS in the test subject.

In a third aspect, the present invention provides a method of diagnosing chronic fatigue syndrome (CFS) in a test subject, said method comprising the steps of:
(i) determining a concentration of L-carnitine in a first body sample from the test subject, and
(ii) determining a concentration of at least one individual fatty acid that corresponds to an acyl group of at least one individual acylcarnitine compound in a second body sample from the test subject, wherein said first and second body samples may be the same, and
(iii) determining a ratio of the concentration of L-carnitine to the concentration of the at least one individual fatty acid, or
   assessing a relationship between the concentration of the L-carnitine and the concentration of the at least one individual fatty acid;
      wherein an aberrant ratio determined in step (iii) or an aberrant relationship assessed in step (iii) is indicative of CFS in the test subject.

In a fourth aspect, the present invention provides a method of treating chronic fatigue syndrome (CFS) in a subject, said method comprising administering an effective amount of a supplement comprising:
at least one acylcarnitine compound selected from short-chain, medium-chain and long-chain acylcarnitines,
L-carnitine, or an acylcarnitine that may be converted within a subject to L-carnitine, in combination with at least one fatty acid selected from short-chain, medium-chain and long-chain fatty acids, or
at least one acylcarnitine in combination with at least one fatty acid selected from short-chain, medium-chain and long-chain fatty acids.

In a fifth aspect, the present invention provides a method of treating chronic fatigue syndrome (CFS) in a subject, said method comprising the steps of:
(i) identifying in said subject a deficiency in one or more individual acylcarnitine compound(s) by
   (a) determining a concentration of at least one individual acylcarnitine in a test body sample from the subject, and
   (b) comparing the concentration determined in (a) to a reference concentration of the at least one individual acylcarnitine determined from an equivalent body sample(s) from a healthy control subject (or a reference concentration range of the at least one individual acylcarnitine determined from equivalent body samples from a plurality of healthy control subjects),
      wherein a lesser concentration of the at least one individual acylcarnitine compound(s) from the subject compared to the reference concentration (or reference concentration range) indicates a deficiency in the said at least one individual acylcarnitine compound(s); and
(ii) administering an effective amount of a supplement comprising the deficient at least one individual acylcarnitine compound(s), L-carnitine (or an acylcarnitine that may be converted within a subject to L-carnitine) in combination with at least one fatty acid that corresponds to the deficient at least one individual acylcarnitine compound(s), or the deficient at least one individual acylcarnitine compound(s) in combination with at least one fatty acid that corresponds to the deficient at least one individual acylcarnitine compound(s).

In a sixth aspect of the present invention, the present invention provides a method of fortifying a food comprising adding to the food a supplement comprising:
at least one acylcarnitine compound selected from short-chain, medium-chain and long-chain acylcarnitines,
L-carnitine, or an acylcarnitine that may be converted within a subject to L-carnitine, in combination with at least one fatty acid selected from short-chain, medium-chain and long-chain fatty acids, or
at least one acylcarnitine in combination with at least one fatty acid selected from short-chain, medium-chain and long-chain fatty acids.

In a seventh aspect, the present invention provides a method of treating chronic fatigue syndrome (CFS) in a subject, said method comprising administering an effective amount of a modulator of carnitine/acylcarnitine metabolism wherein the modulator stimulates the activity of an enzyme selected from the group consisting of carnitine palmitoyltransferase (CPT)-I, carnitine palmitoyltransferase (CPT)-II and carnitine/acylcarnitine translocase.

### BRIEF DESCRIPTION OF THE FIGURES

FIG. 1 provides a schematic representation that illustrates the role of L-carnitine, acylcarnitine, Coenzyme A (CoA), carnitine palmitoyltransferase (CPT)-I, CPT-II and carnitine/acylcarnitine translocase in fatty acid γ-oxidation; wherein the transfer of the acyl group from a fatty acid to L-carnitine to produce an individual acylcarnitine is referred to as an acyltransferase reaction; and

FIG. 2 provides a graph showing endogenous plasma oleyl-L-carnitine (C18:1) and linoleyl-L-carnitine (C18:2) concentrations (μmol/L) in CFS patients (closed circles) and healthy control subjects (open circles).

### DETAILED DESCRIPTION OF THE INVENTION

It is thought that fatty acid metabolism may be linked to CFS; however, studies in this area have produced conflicting results. It has now been found that particular individual acylcarnitines are present in modified concentrations (ie decreased or increased concentrations) in CFS patients compared to healthy control subjects. Further, it has been realised that this finding may provide a means to diagnose CFS and/or provide the basis for the rational design of novel methods for treatment of CFS.

The term “fatty acid” as used herein will be understood by persons skilled in the art as referring to a carboxylic acid, represented by the formula R—C(═O)OH, wherein the R represents an alkyl group. The alkyl group, together with the carbon atom from the carboxylic group, is referred to as a carbon chain. The carbon chain may be of variable length, for example, between 4 and 32 (or more) carbon atoms. A “short [carbon] chain” is considered to be a chain with less than 6 carbon atoms but, preferably, no less than 4 carbon atoms; a “medium chain” is considered to be a chain with 6 to 11 carbon atoms; and a “long chain” is considered to be a chain with 12 or more carbon atoms. The term “very long chain” is sometimes used for chains with more than 22 carbon atoms; however, the term “long chain” is used herein when referring to any chain with 12 or more carbon atoms. The chains are generally linear, and may be branched or unbranched. The chains can be “saturated”, meaning that the carbon atoms are connected by single bonds only, or may be “unsaturated”, meaning that there is at least one double bond (or triple bond) between the carbon atoms. The “acyl group” of a fatty acid has the formula R—C(═O)—, wherein R represents an alkyl group. Different fatty acids have different alkyl groups and hence different acyl groups.

There are several nomenclature systems assigned to fatty acids, for example the “trivial nomenclature” (or common name) system and the “lipid number” system, both of which are used herein. The lipid number system takes the form C:D, where C is the number of carbon atoms in the fatty acid, and D is the number of double bonds in the fatty acid. For example, oleic acid has the formula CH₃(CH₂)₇CH═CH(CH₂)₇COOH. It has 18 carbon atoms, and one double bond, and so is given the lipid number 18:1. However, the lipid number system can be ambigtious as different fatty acids can have the same lipid number, if, for example, a double bond is present in a different place on a chain that has the same number of carbon atoms. The lipid number system may also utilise “DC”, wherein the DC signifies that the compound is dicarboxylic; that is, the compound has two carboxylic acid groups.

The term “individual acylcarnitine” will be understood by persons skilled in the art to refer to a molecule consisting of L-carnitine to which the acyl group of a particular fatty acid is bound. The acylcarnitine is accordingly assigned the same lipid number as the corresponding fatty acid; however, it is preceded by a “C”, representing L-carnitine. It will also be understood by persons skilled in the art that, in the context of the present invention, the individual acylcarnitine(s) may be regarded as “endogenous” since they arise from L-carnitine and acylcarnitine homeostasis processes within a subject. As such, the individual acylcarnitine may be, for example, acetyl-L-carnitine (C2); propionyl-L-carnitine (C3); malonyl-L-carnitine (C3DC); butyryl-L-carnitine (C4); hydroxy-butyryl-L-carnitine (C4-OH); succinyl-L-carnitine (C4DC); isovaleryl-L-carnitine (C5); tiglyl-L-carnitine (C5:1); hydroxy-isovaleryl-L-carnitine (C5-OH); glutaryl-L-carnitine (C5DC); hexanoyl-L-carnitine (C6); hexenoyl-L-carnitine (C6:1); adipyl-L-carnitine (C6DC); octanoyl-L-carnitine (C8); octenoyl-L-carnitine (C8:1); suberyl-L-carnitine (C8DC); decanoyl-L-carnitine (C10); decenoyl-L-carnitine (C10:1); decadienoyl-L-carnitine (C10:2); sebacyl-L-carnitine (C10DC); lauroyl-L-carnitine (C12); dodecenoyl-L-carnitine (C12:1); dodecanedioyl-L-carnitine (C12DC); myristoyl-L-carnitine (C14); myristoleyl-L-carnitine C14:1); tetradecadienoyl-L-carnitine (C14:2); hydroxy-myristoyl-L-carnitine (C14-OH); palmitoyl-L-carnitine (C16); palmitoleyl-L-carnitine (C16:1); hydroxyl-palmitoyl-L-carnitine (C16-OH); hydroxy-palmitoleyl-L-carnitine (C16:1-OH); stearoyl-L-carnitine (C18); oleyl-L-carnitine (C18:1); linoleyl-L-carnitine (C18:2); and hydroxy-oleyl-L-carnitine (C18:1-OH).

In a first aspect, the present invention provides a method of diagnosing chronic fatigue syndrome (CFS) in a test subject, said method comprising the steps of:
(i) determining a concentration of at least one individual acylcarnitine compound in a body sample from the test subject; and
(ii) comparing the concentration determined in step (i) to a reference concentration of the at least one individual acylcarnitine determined from an equivalent body sample from a healthy control subject (or a reference concentration range of the at least one individual acylcarnitine determined from equivalent body samples from a plurality of healthy control subjects),
   wherein a difference in the concentration of the at least one individual acylcarnitine from the test subject compared to the reference concentration (or reference concentration range) is indicative of CFS in the test subject.

Preferably, the at least one individual acylcarnitine is a medium-chain or a long-chain acylcarnitine. For example, the at least one acylcarnitine may have a carbon chain that is 6 or more carbon atoms long. Preferably, the acylcarnitine has a carbon chain that is 12 or more carbon atoms long.

In an embodiment, the at least one individual acylcarnitine is selected from the group consisting of octenoyl-L-carnitine, dodecanedioyl-L-carnitine, myristoyl-L-carnitine, palmitoleyl-L-carnitine, stearoyl-L-carnitine, oleyl-L-carnitine, linoleyl-L-carnitine and hydroxyl-oleyl-L-carnitine. More preferably, the at least one individual acylcarnitine is selected from oleyl-L-carnitine and linoleyl-L-carnitine.

The method of the first aspect does not require the use of a detectably-labelled acylcarnitine, since the at least one individual acylcarnitine referred to in step (i) is endogenous; that is, the at least one individual acylcarnitine is found naturally in the subject, having arisen from L-carnitine and acylcarnitine homeostasis processes and/or dietary sources.

A diagnosis of CFS may be made, for example, when the concentration of at least one individual acylcarnitine from the test subject is decreased compared to that of the reference concentration (or reference concentration range), wherein the at least one individual acylcarnitine is selected from the group consisting of octenoyl-L-carnitine, myristoyl-L-carnitine, palmitoleyl-L-carnitine, stearoyl-L-carnitine, oleyl-L-carnitine and linoleyl-L-carnitine.

However, in another example, a diagnosis of CFS may be made when the concentration of the at least one individual acylcarnitine from the test subject is increased compared to that of the reference concentration (or reference concentration range), wherein the at least one individual acylcarnitine is selected from dodecanedioyl-L-carnitine and hydroxyl-oleyl-L-carnitine.

In an embodiment of the method of the first aspect, the method comprises the steps of:
(i) determining the concentration of two or more individual acylcarnitine compounds in a body sample from the test subject; and
(ii) comparing the concentrations determined in step (i) to reference concentrations of the two or more individual acylcarnitines determined from an equivalent body sample(s) from a healthy control subject (or reference concentration ranges of the two or more individual acylcarnitines determined from equivalent body samples from a plurality of healthy control subjects),
   wherein a difference in the concentrations of the two or more individual acylcarnitines from the test subject compared to the reference concentrations (or reference concentration ranges) is indicative of CFS in the test subject.

Preferably, the two or more individual acylcarnitines are selected from those listed above; however, persons skilled in the art will appreciate that other acylcarnitine compounds may also be suitable. In some embodiments, the two or more individual acylcarnitines will be three or more, four or more, or five or more, etc, individual acylcarnitines.

The concentration of the individual acylcarnitine(s) from the test subject may be compared to the concentration of the same individual acylcarnitine(s) from an equivalent body sample(s) from a healthy control subject, or, preferably, to a concentration range of the same acylcarnitine(s) from equivalent body samples from a plurality of healthy control subjects (eg 10 to 1000 healthy control subjects). The body samples may be any body sample type that can be sampled for acylcarnitine concentration. For example, the body samples may be whole blood, serum, plasma, urine or sputum. Preferably, body samples are plasma, serum or whole blood.

The concentration of the individual acylcarnitine(s) in the body samples may be determined by any suitable method including those well known to persons skilled in the art including mass spectrometry (eg tandem mass spectrometry); chromatographic techniques, such as high performance liquid chromatography (eg radioisotopic exchange HPLC), gas chrorhatography and thin layer chromatography; electrochemical sensing; and chemical sensing using suitable probes, etc.

It has also been realised that a diagnosis of CFS in a subject may be based upon the determination of an aberrant concentration of at least one acylcarnitine or L-carnitine present in a test body sample(s) relative to the concentration of at least one fatty acid corresponding to an acyl group of at least one acylcarnitine compound and, similarly, an aberrant relationship between the concentration of at least one acylcarnitine or L-carnitine present in a test body sample(s) and the concentration of at least one fatty acid corresponding to an acyl group of at least one acylcarnitine compound.

Thus, in a second aspect, the present invention provides a method of diagnosing chronic fatigue syndrome (CFS) in a test subject, said method comprising the steps of:
(i) determining a concentration of at least one individual acylcarnitine compound in a first body sample from the test subject,
(ii) determining a concentration of at least one individual fatty acid that corresponds to an acyl group of said at least one individual acylcarnitine compound in a second body sample from the test subject, wherein said first and second body samples may be the same, and
(iii) determining a ratio of the concentration of the at least one individual acylcarnitine compound to the concentration of the at least one individual fatty acid, or
   assessing a relationship between the concentration of the at least one individual acylcarnitine compound and the concentration of the at least one individual fatty acid;
      wherein an aberrant ratio determined in step (iii) or an aberrant relationship assessed in step (iii) is indicative of CFS in the test subject.

Preferably, step (iii) comprises determining a ratio of the concentration of the at least one individual acylcarnitine compound to the concentration of the at least one individual fatty acid, in which case, the determination of an aberrant ratio is indicative of CFS in the test subject. An aberrant ratio in this context may, for example, constitute a fatty acid:acylcarnitine concentration ratio that differs from a reference ratio (eg a control ratio) determined from one or more healthy subjects by ≧1.5 fold, more preferably ≧two-fold, and even more preferably ≧three-fold.

Alternatively, step (iii) comprises assessing a relationship between the concentration of the at least one individual acylcarnitine compound and the concentration of the at least one individual fatty acid, in which case, the assessment of an aberrant relationship is indicative of CFS in the test subject.

In a third aspect, the present invention provides a method of diagnosing chronic fatigue syndrome (CFS) in a test subject, said method comprising the steps of:
(i) determining a concentration of L-carnitine in a first body sample from the test subject, and
(ii) determining a concentration of at least one individual fatty acid that corresponds to an acyl group of at least one individual acylcarnitine compound in a second body sample from the test subject, wherein said first and second body samples may be the same, and
(iii) determining a ratio of the concentration of L-carnitine to the concentration of the at least one individual fatty acid, or
   assessing a relationship between the concentration of the L-carnitine and the concentration of the at least one individual fatty acid;
      wherein an aberrant ratio determined in step (iii) or an aberrant relationship assessed in step (iii) is indicative of CFS in the test subject.

Preferably, step (iii) comprises determining a ratio of the concentration of L-carnitine and the concentration of the at least one individual fatty acid, in which case, the determination of an aberrant ratio is indicative of CFS in the test subject. An aberrant ratio in this context may, for example, constitute a fatty acid: L-carnitine concentration ratio that differs from a reference ratio (eg a control ratio) determined from one or more healthy subjects by ≧1.5 fold, more preferably ≧two-fold, and even more preferably ≧three-fold.

Alternatively, step (iii) comprises assessing a relationship between the concentration of L-carnitine and the concentration of the at least one individual fatty acid, in which case, the assessment of an aberrant relationship is indicative of CFS in the test subject.

In the method of the second and third aspects, the first and second body samples are preferably the same. That is, preferably, the method utilises a single sample (or aliquots of a single sample) in the determination of the concentrations mentioned in the respective steps (i) and (ii). The sample may therefore be a single sample of whole blood, serum, plasma, urine or sputum.

The concentration of the individual acylcarnitine(s) and L-carnitine, in the case of the method of the third aspect, and the individual fatty acid(s) in the body samples may be determined by any suitable method such as those mentioned above in respect of the method of the first aspect.

The methods of the second and third aspects do not require the use of a detectably-labelled acylcarnitine, L-carnitine or fatty acid, since the at least one individual acylcarnitine, L-carnitine or at least one individual fatty acid referred to therein is endogenous; that is, the at least one individual acylcarnitine, L-carnitine or at least one individual fatty acid are found naturally in the subject, having arisen from L-carnitine and acylcarnitine homeostasis processes and/or dietary sources.

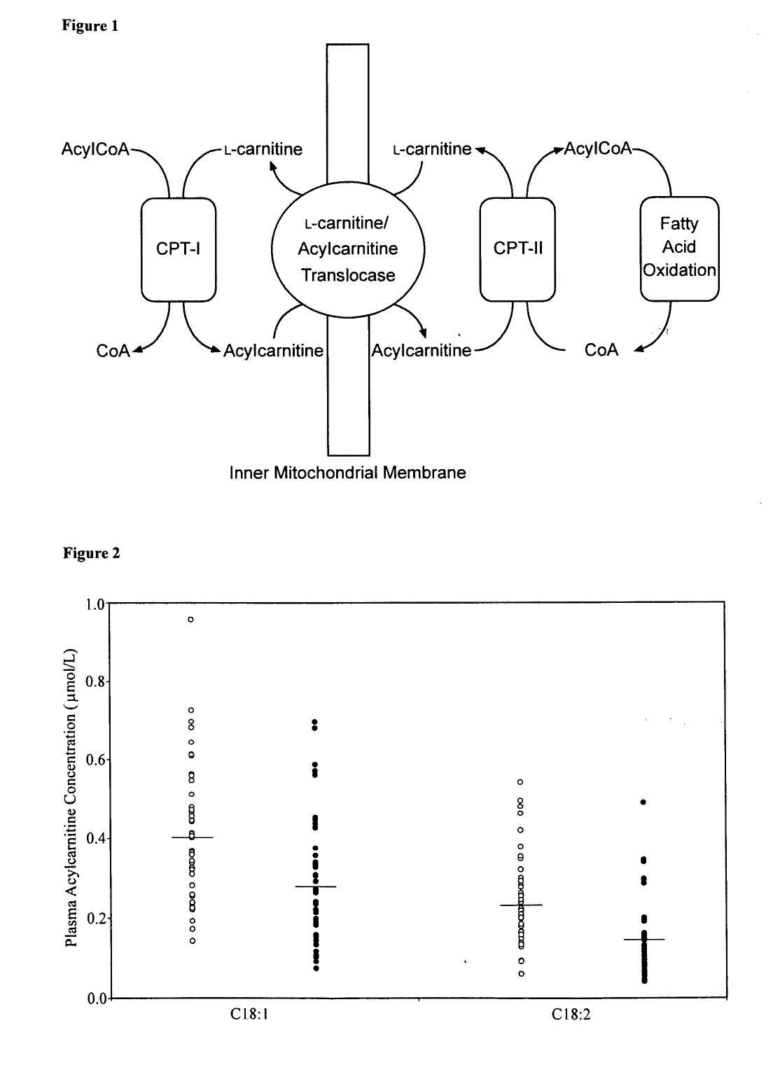

It has been additionally realised that the modified concentration of individual acylcarnitines in CFS patients compared to healthy subjects may be at least partly associated with at least some of the symptoms of CFS, for example, a decreased concentration of an individual acylcarnitine may be associated with fatigue due to a lesser amount of the acylcarnitine being available for energy metabolism compared to healthy subjects. Accordingly, supplementing a CFS patient with an individual acylcarnitine may reduce at least some of the CFS symptoms. Alternatively or additionally, administering a patient with L-carnitine (or an acylcarnitine such as acetyl-L-carnitine (ALC) or propionyl-L-carnitine (PLC) that may be converted within a subject to L-carnitine) and an individual fatty acid may provide a means to increase the concentration of the corresponding acylcarnitine via the acyltransferase reaction shown in FIG. 1. Similarly, supplementing a patient with an individual acylcarnitine in combination with at least one individual fatty acid may also increase the concentration of the corresponding acylcarnitine within a CFS patient.

Thus, in a fourth aspect, the present invention provides a method of treating chronic fatigue syndrome (CFS) in a subject, said method comprising administering an effective amount of a supplement comprising:
at least one acylcarnitine compound selected from short-chain, medium-chain and long-chain acylcarnitines,
L-carnitine, or an acylcarnitine that may be converted within a subject to L-carnitine, in combination with at least one fatty acid selected from short-chain, medium-chain and long-chain fatty acids, or
at least one acylcarnitine in combination with at least one fatty acid selected from short-chain, medium-chain and long-chain fatty acids.

Preferably, the carbon chain of the acylcarnitine and/or the fatty acid is 12 or more carbon atoms long.

For example, the at least one acylcarnitine may be selected from the group consisting of octenoyl-L-carnitine, dodecanedioyl-L-carnitine, myristoyl-L-carnitine, palmitoleyl-L-carnitine, stearoyl-L-carnitine, oleyl-L-carnitine, linoleyl-L-carnitine and hydroxyl-oleyl-L-carnitine. However, preferably, the at least one acylcarnitine is selected from oleyl-L-carnitine and linoleyl-L-carnitine.

Similarly, the at least one individual fatty acid may be selected from the group consisting of octenoic acid, dodecanedioic acid, myristoic acid, palmitoleic acid, stearoic acid, oleic acid, linoleic acid and hydroxyl-oleic acid. However, preferably, the at least one individual fatty acid is selected from oleic acid and linoleic acid.

In an embodiment, the method of treating CFS in a subject comprises administering an effective amount of a supplement comprising two or more individual acylcarnitine compounds wherein at least one of the individual acylcarnitines is selected from medium-chain and long-chain acylcarnitines, or a supplement comprising L-carnitine (or an acylcarnitine such as ALC or PLC that may be converted within a subject to L-carnitine) in combination with two or more individual fatty acids wherein at least one of the individual fatty acids is selected from medium-chain and long-chain fatty acids.

It has also been realised that CFS patients may be deficient in a particular individual acylcarnitine if the patient has a decreased ability to convert L-carnitine and the corresponding individual fatty acid to the individual acylcarnitine. Therefore, by administering to the patient a supplement that modulates carnitine/acylcarnitine metabolism, for example, by modulation of the activity or expression levels of carnitine palmitoyltransferase (CPT)-I (ie the enzyme that catalyses the transfer of an acyl group of a fatty acid to L-carnitine to form the individual acylcarnitine) and/or carnitine palmitoyltransferase (CPT)-II (ie the enzyme that catalyses the transfer of the acyl group from the L-carnitine molecule to a mitochondrial CoA) and/or carnitine/acylcarnitine translocase (ie the enzyme responsible for transporting both carnitine and acylcarnitines into and out of the mitochondria, across the inner mitochondrial membrane), the benefits of supplementing the patient with an individual fatty acid may be enhanced.

Accordingly, in an embodiment of the method of the fourth aspect, the method further comprises administering a modulator(s) of any one or more of CPT-I, CPT-II and carnitine/acylcarnitine translocase. More preferably, the modulator(s) stimulates the activity of at least CPT-I. The modulator(s) may be a drug or a dietary supplement. For instance, L-carnitine (Yoon et al., 2003) and all-trans retinoic acid (Amengual et al., 2008) have been shown to upregulate CPT-I expression or activity. Other suitable modulators include omega-3 fatty acids such as eicospentaenoic acid (EPA; C20:5) and docosahexanoic acid (DHA; C22:6), which may either be provided in substantially pure compound form or as a mixture such as, conveniently, a fish oil preparation.

Accordingly, the modulator(s) is preferably selected from the group consisting of L-carnitine (or an acylcarnitine that may be converted within a subject to L-carnitine), all-trans retinoic acid, fatty acids (particularly, omega-3 fatty acids) and combinations thereof.

The modulator(s) of CPT-I and/or CPT-II and/or carnitine/acylcarnitine translocase may be administered before or after the supplement, however preferably, the supplement itself comprises the CPT-I/CPT-II/carnitine/acylcarnitine translocase modulator(s).

Thus, in a particular embodiment of the method of the fourth aspect, the method comprises administering an effective amount of a supplement comprising L-carnitine (or an acylcarnitine that may be converted within a subject to L-carnitine), in combination with at least one fatty acid selected from short-chain, medium-chain and long-chain fatty acids (eg oleic acid and/or linolenic acid) and an omega-3 fatty acid (eg EPA and/or DHA). In such a supplement, the relative amounts of the components may be:

| | |
|---|---|
| L-carnitine (or an acylcarnitine that may | 60 to 95 wt % |
| be converted to L-carnitine), | |
| short-chain, medium-chain or long-chain | 0.5 to 20 wt % |
| fatty acid | |
| omega-3 fatty acid | 0.5 to 20 wt % |

Where the supplement administered in the method of the fourth aspect comprises an acylcarnitine that may be converted within a subject to L-carnitine, preferably that acylcarnitine is PLC. PLC may offer the advantage of additionally enhancing energy metabolism through an anaplerotic mechanism via the generation of succinyl-CoA, a substrate for the Krebs cycle (Brevetti et al., 1997).

The supplement may further comprise a pharmaceutically-acceptable carrier, excipient and/or diluent.

The “effective amount” of the supplement will be any amount that will elicit a beneficial or therapeutic effect in the subject. However, generally, the effective amount will be about 0.01 to about 500 mg/kg of the subject body weight per day which can be administered in single or multiple doses. Preferably, the amount will be about 0.1 to about 250 mg/kg per day; more preferably, about 0.5 to about 100 mg/kg per day.

The supplement may be administered to the subject by any suitable means, for example, orally, intravenously, intramuscularly or intranasally. However, preferably, the supplement is administered orally. Accordingly, the supplement is preferably formulated in an oral dosage form such as, for example, a capsule, tablet, caplet, granules or powders (which may be suspended or dissolved in water to provide a beverage). In some embodiments, the supplement is provided to the subject in a fortified food as described in more detail below.

In a fifth aspect, the present invention provides a method of treating chronic fatigue syndrome (CFS) in a subject, said method comprising the steps of:
(i) identifying in said subject a deficiency in one or more individual acylcarnitine compound(s) by
   (a) determining a concentration of at least one individual acylcarnitine in a body sample from the subject, and
   (b) comparing the concentration determined in (a) to a reference concentration of the at least one individual acylcarnitine determined from an equivalent body sample(s) from a healthy control subject (or a reference concentration range of the at least one individual acylcarnitine determined from equivalent body samples from a plurality of healthy control subjects),
      wherein a lesser concentration of the at least one individual acylcarnitine compound(s) from the subject compared to the reference concentration (or reference concentration range) indicates a deficiency in the said at least one individual acylcarnitine compound(s); and
(ii) administering an effective amount of a supplement comprising the deficient at least one individual acylcarnitine compound(s), L-carnitine (or an acylcarnitine that may be converted within a subject to L-carnitine) in combination with at least one fatty acid that corresponds to the deficient at least one individual acylcarnitine compound(s), or the deficient at least one individual acylcarnitine compound(s) in combination with at least one fatty acid that corresponds to the deficient at least one individual acylcarnitine compound(s).

The concentration of the individual acylcarnitine(s) from the subject may be compared to the concentration of the same individual acylcarnitine from an equivalent body sample(s) from a healthy control subject, or, preferably, from a concentration range of the same acylcarnitine(s) from equivalent body samples from healthy control subjects. The body samples may be any body sample type that can be sampled for acylcarnitine concentration. For example, the body samples may be whole blood, serum, plasma, urine or sputum. Preferably, the body samples are plasma, serum or whole blood.

The method of the fifth aspect, like that of the first aspect, does not require the use of a detectably-labelled acylcarnitine.

The phrase “[at least one] individual fatty acid that corresponds to the deficient [at least one] individual acylcarnitine” is intended to refer to a particular individual fatty acid that has the same acyl group as the particular individual acylcarnitine that is deficient in the CFS patient. In other words, the corresponding fatty acid is a particular individual fatty acid that could theoretically be transformed into the particular individual acylcarnitine (that has a decreased concentration in the CFS patient) by CPT-I as shown in FIG. 1. For example, octenoic acid is the individual fatty acid that corresponds to the individual acylcarnitine octenoyl-L-carnitine; and similarly, dodecanedioic acid corresponds to dodecanedioyl-L-carnitine; myristoic acid corresponds to myristoyl-L-carnitine; palmitoleic acid corresponds to palmitoleyl-L-carnitine; stearoic acid corresponds to stearoyl-L-carnitine; oleic acid corresponds to oleyl-L-carnitine; linoleic acid corresponds to linoleyl-L-carnitine; and hydroxyl-oleic acid corresponds to hydroxyl-oleyl-L-carnitine; etc.

It will be understood by persons skilled in the art that in some embodiments the supplement may comprise L-carnitine (or an acylcarnitine such as ALC or PLC that may be converted within a subject to L-carnitine) in combination with two or more individual fatty acids that correspond to two or more individual acylcarnitines as described below. For example, oleic acid and linoleic acid are the fatty acids that correspond to oleyl-L-carnitine and linoleyl-L-carnitine, respectively.

Preferably, the at least one individual acylcarnitine is a medium-chain or a long-chain acylcarnitine. For example, the at least one acylcarnitine may have a carbon chain that is 6 or more carbon atoms long. Preferably, the acylcarnitine has a carbon chain that is 12 or more carbon atoms long. In an embodiment, the at least one individual acylcarnitine is selected from the group consisting of octenoyl-L-carnitine, dodecanedioyl-L-carnitine, myristoyl-L-carnitine, palmitoleyl-L-carnitine, stearoyl-L-carnitine, oleyl-L-carnitine, linoleyl-L-carnitine and hydroxyl-oleyl-L-carnitine. More preferably, the at least one individual acylcarnitine is selected from oleyl-L-carnitine and linoleyl-L-carnitine.

Accordingly, the at least one individual fatty acid may be selected from the group consisting of octenoic acid, dodecanedioic acid, myristoic acid, palmitoleic acid, stearoic acid, oleic acid, linoleic acid and hydroxyl-oleic acid. Preferably, the individual fatty acid(s) is selected from oleic acid and linoleic acid.

In an embodiment of the method of the fifth aspect, the method further comprises administering a modulator(s) of any one or more of CPT-I, CPT-II and carnitine/acylcarnitine translocase. More preferably, the modulator(s) stimulates the activity of at least CPT-I. The modulator(s) may, for example, be selected from the group consisting of L-carnitine (or an acylcarnitine that may be converted within a subject to L-carnitine), all-trans retinoic acid, fatty acids (particularly, omega-3 fatty acids) and combinations thereof.

In some embodiments, the supplement administered in the methods of the fourth and fifth aspects is provided to the subject in a fortified food. The fortified food may be any suitable food that is able to be modified to contain the supplement in a desired amount. For example, the fortified food may be bread, cake, biscuits (crackers or cookies), cereal, food bars (such as health food bars and muesli bars), drinks, etc.

In a sixth aspect, the present invention provides a method of fortifying a food comprising adding to the food a supplement comprising:
at least one acylcarnitine compound selected from short-chain, medium-chain and long-chain acylcarnitines,
L-carnitine, or an acylcarnitine that may be converted within a subject to L-carnitine, in combination with at least one fatty acid selected from short-chain, medium-chain and long-chain fatty acids, or
at least one acylcarnitine in combination with at least one fatty acid selected from short-chain, medium-chain and long-chain fatty acids.

In an embodiment, the method further comprises fortifying the food with a modulator(s) of any one or more of CPT-I, CPT-II and carnitine/acylcarnitine translocase. More preferably, the modulator(s) stimulates the activity of at least CPT-I. The modulator(s) may, for example, be selected from the group consisting of L-carnitine (or an acylcarnitine that may be converted within a subject to L-carnitine), all-trans retinoic acid, fatty acids (particularly, omega-3 fatty acids) and combinations thereof. Fortifying the food with the modulator(s) can be conveniently achieved by including the modulator(s) in the said supplement.

The supplement may be added to the food in any suitable manner, for example, the supplement may be added during the mixing process of foods, or may alternatively be added following baking of the food product, or alternatively, added prior to packaging.

The invention further extends to a fortified food produced in accordance with the method of the sixth aspect.

In a seventh aspect, the present invention provides a method of treating chronic fatigue syndrome (CFS) in a subject, said method comprising administering an effective amount of a modulator of carnitine/acylcarnitine metabolism, for example, a modulator of carnitine palmitoyltransferase (CPT)-I and/or carnitine palmitoyltransferase (CPT)-II and/or carnitine/acylcarnitine translocase.

While not wishing to be bound by theory, it is considered that the administration of a modulator(s) which stimulates the activity of at least CPT-I will represent an effective treatment of CFS by modulating carnitine and/or fatty acid metabolism so as to increase the ratio of acylcarnitines to free fatty acids.

The modulator(s) may be a drug or a dietary supplement. Preferably, the modulator(s) is selected from the group consisting of L-carnitine (or an acylcarnitine that may be converted within a subject to L-carnitine), all-trans retinoic acid, fatty acids (particularly, omega-3 fatty acids) and combinations thereof. More preferably, the modulator(s) comprises L-carnitine (or an acylcarnitine that may be converted within a subject to L-carnitine) in combination with one or more omega-3 fatty acids such as EPA and DHA, which may either be provided in substantially pure compound form or as a mixture such as, conveniently, a fish oil preparation. In such a combination, the relative amounts of the components may be:

| | |
|---|---|
| L-carnitine (or an acylcarnitine that may | 60 to 95 wt % |
| be converted to L-carnitine) | |
| omega-3 fatty acid | 1 to 40 wt % |

The “effective amount” of the modulator(s) will be any amount that will elicit a beneficial or therapeutic effect in the subject. However, generally, the effective amount will be about 0.01 to about 500 mg/kg of the subject body weight per day which can be administered in single or multiple doses. Preferably, the amount will be about 0.1 to about 250 mg/kg per day; more preferably, about 0.5 to about 100 mg/kg per day.

The modulator(s) may be administered to the subject by any suitable means, for example, orally, intravenously, intramuscularly or intranasally. However, preferably, the modulator(s) is administered orally. Accordingly, the modulator(s) is preferably formulated in an oral dosage form such as, for example, a capsule, tablet, caplet, granules or powders (which may be suspended or dissolved in water to provide a beverage). The modulator(s) may be provided in combination with a pharmaceutically-acceptable carrier, excipient and/or diluent.

The invention is hereinafter described by reference to the following non-limiting example and accompanying figures.

### EXAMPLE

### Example 1

Previous studies have predominantly used enzymatic assays for the quantification of L-carnitine, total carnitine and total acylcarnitine levels. Only one previous study has investigated individual acylcarnitine levels in CFS patients (Soetekouw et al., 2000). This study reported no significant differences in individual acylcarnitine levels between CFS patients and healthy controls; however, only a limited number of individual acylcarnitines were quantified and the experimental design further limited the usefulness of the data obtained.

Tandem mass spectrometry methods have been developed which are capable of quantifying individual acylcarnitine levels in human plasma (Chace et al., 1997; Chace et al., 2003), and this method has now been utilised to provide a more complete representation of the full carnitine profile. The present study examined the concentration of endogenous plasma L-carnitine and a complement of individual acylcarnitines in CFS patients compared with age- and gender-matched healthy controls. The aim of this study was to quantify endogenous plasma L-carnitine and 35 individual acylcarnitines in CFS patients compared to age- and gender-matched healthy controls.

### Methods and Materials

### Study Design

Chronic fatigue syndrome patients (n=44) were recruited via the Chronic Fatigue Syndrome Society of South Australia (ME/CFS Society [SA] Inc). Patients had been previously diagnosed with CFS by a physician according to the standard diagnostic criteria, that is, fatigue and at least four other symptoms as described in Table 1. Age- and gender-matched healthy subjects with no significant illnesses (n=49), were recruited from the general population via advertising. Neither patients nor healthy subjects had received any carnitine supplementation in the two months prior to the assessment.

TABLE 1
Diagnostic criteria of chronic fatigue syndrome*
Fatigue
Clinically-evaluated, unexplained, persistent or relapsing fatigue persistent
for six months or more, that:
is of new or definite onset;
is not the result of ongoing exertion;
is not substantially alleviated by rest; and
results in substantial reduction in previous levels of occupational,
educational, social or personal activities
AND
Other Symptoms
Four or more of the following symptoms that are concurrent, persistent for
six months or more and which did not predate the fatigue:
impaired short-term memory or concentration
sore throat
tender cervical or axillary lymph nodes
muscle pain
multi-joint pain without arthritis
headaches of a new type, pattern, or severity
unrefreshing sleep
post-exertional malaise lasting more than 24 hours
*Royal Australasian College of Physicians Working Group (2002)

### Fatigue Severity Scale

On the study assessment day, each subject/patient completed a Fatigue Severity Scale questionnaire and had a single blood sample collected via venepuncture for carnitine profiling. The Fatigue Severity Scale is a validated functional measure which comprises nine items that are rated according to a Likert-type rating scale from 1 to 7, with 1 indicating no impairment and 7 indicating severe impairment (Table 2; Krupp et al., 1989). The Fatigue Severity Scale has been shown to be an appropriate and accurate measure of fatigue severity and symptomology, and is able to distinguish between individuals with chronic fatigue syndrome-like symptomology and those individuals with no of varying levels of general fatigue (Taylor et al., 2000).

### Carnitine Profiling

A single blood sample was collected from each study subject to determine the plasma concentration of various carnitine and acylcarnitine types (described below). Analysis was conducted using a MDS-SCIEX API4000 triple quadruple tandem mass spectrometer (Applied Biosystems Inc, Foster City, Calif., United States of America) with sample delivery using a 1100 HPLC system (Agilent Technologies, Santa Clara, Calif., United States of America). Aliquots (2 μL) of each plasma sample were applied to 3 mm punches of filter paper (Whatman BFC-180, Whatman Inc, Fairfield, N.J., United States of America) and allowed to dry at room temperature. Once dry, filter papers were shipped to the analytical laboratory for analysis.

A solution of pure methanol containing known concentrations of stable isotopically enriched acylcarnitines was used to extract samples from the filter paper as described below.

Samples were extracted from the filter paper using the solution of pure methanol containing the known concentrations of stable isotopically enriched acylcarnitines. After a 15 minute extraction period, samples were dried under nitrogen. Samples were then esterified using acidified butanol to form the butyl-ester of each acylcarnitine followed by drying under nitrogen to remove excess butanolic HCl. The butyl-esters were determined by precursor scan of 85.1 amu. The levels of acylcarnitines were determined against the respective deuterated stable isotope using Analyst® software (Applied Biosystems Inc).

TABLE 2
Fatigue Severity Scale (Krupp et al., 1989)
Indicate your agreement/disagreement with the following statements
on a scale of 1 to 7, with a score of 1 indicating STRONGLY DISAGREE
and a score of 7 indicating STRONGLY AGREE.

| | | |
|---|---|---|
| 1. | My motivation is lower when I am fatigued. | 1 2 3 4 5 6 7 |
| 2. | Exercise brings on my fatigue. | 1 2 3 4 5 6 7 |
| 3. | I am easily fatigued. | 1 2 3 4 5 6 7 |
| 4. | Fatigue interferes with my physical functioning. | 1 2 3 4 5 6 7 |
| 5. | Fatigue causes frequent problems for me. | 1 2 3 4 5 6 7 |
| 6. | My fatigue prevents sustained physical functioning. | 1 2 3 4 5 6 7 |
| 7. | Fatigue interferes with me carrying out certain | 1 2 3 4 5 6 7 |
| | duties and responsibilities. | |
| 8. | Fatigue is among my three most disabling symptoms. | 1 2 3 4 5 6 7 |
| 9. | Fatigue interferes with my work, family or social life. | 1 2 3 4 5 6 7 |

### Carnitine and Acylcarnitines Detected

A single blood sample was collected from each study subject to determine the plasma concentration of the following analytes: L-carnitine (LC); total carnitine (TC; ie all carnitine including all of the acylcarnitines); total acylcarnitine (AcylLC); acetyl-L-carnitine (C2); propionyl-L-carnitine (C3); malonyl-L-carnitine (C3DC); butyryl-L-carnitine (C4); hydroxy-butyryl-L-carnitine (C4-OH); succinyl-L-carnitine (C4DC); isovaleryl-L-carnitine (C5); tiglyl-L-carnitine (C5:1); hydroxy-isovaleryl-L-carnitine (C5-OH); glutaryl-L-carnitine (C5DC); hexanoyl-L-carnitine (C6); hexenoyl-L-carnitine (C6:1); adipyl-L-carnitine (C6DC); octanoyl-L-carnitine (C8); octenoyl-L-carnitine (C8:1); suberyl-L-carnitine (C8DC); decanoyl-L-carnitine (C 10); decenoyl-L-carnitine (C10:1); decadienoyl-L-carnitine (C10:2); sebacyl-L-carnitine (C10DC); lauroyl-L-carnitine (C12); dodecenoyl-L-carnitine (C12:1); dodecanedioyl-L-carnitine (C12DC); myristoyl-L-carnitine (C14); myristoleyl-L-carnitine C14:1); tetradecadienoyl-L-carnitine (C14:2); hydroxy-myristoyl-L-carnitine (C14-OH); palmitoyl-L-carnitine (C16); palmitoleyl-L-carnitine (C16:1); hydroxyl-palmitoyl-L-carnitine (C16-OH); hydroxy-palmitoleyl-L-carnitine (C16:1-OH); stearoyl-L-carnitine (C18); oleyl-L-carnitine (C18:1); linoleyl-C-carnitine (C18:2); and hydroxy-oleyl-L-carnitine (C18:1-OH).

The total acylcarnitine concentration (AcylLC) was determined as the sum of all individual acylcarnitine concentrations; and the total carnitine concentration (TC) was determined as the sum of L-carnitine (LC) and total acylcarnitine (AcylLC) concentrations. It should be noted that, due to the nature of the tandem mass spectrometry method, some assay results represent the sum of two or three carnitine esters. Specifically, C4 represents the sum of two structural isomers with a 4 carbon-chain acyl group: butyryl-L-carnitine and isobutyryl-L-carnitine; C4DC represents the sum of succinyl-L-carnitine and 2-methylmalonyl-L-carnitine; C5 represents the sum of isovaleryl-L-carnitine, valeryl-L-carnitine and 2-methylbutyryl-L-carnitine; C5:1 represents the sum of tiglyl-L-carnitine and pentenoyl-L-carnitine; and C5-OH represents the sum of hydroxyl-isovaleryl-L-carnitine and hydroxyl-valeryl-L-carnitine.

### Statistical Analysis

Unless otherwise indicated, data are expressed as mean±standard deviation. Carnitine concentrations and demographic characteristics (ie age and Fatigue Severity Scale results) obtained from CFS patients were statistically compared to those obtained from healthy subjects using an analysis of variance (ANOVA). Gender distribution between the groups was compared using Pearson's Chi-Squared (χ2) cross-tabulation analysis. Significance was set at an α-level of 0.05. WinNonlin® Professional Version 5.2 (Pharsight Corporation, Mountain View, Calif., United States of America) was used for ANOVA analysis. SPSS for Windows Version 16.0 (SPSS Inc, Chicago, Ill., United States of America) was used for the Pearson's Chi-Squared (χ2) cross-tabulation analysis.

### Results

Forty-four CFS patients (17 males; 27 females), with an average age of 49.9±15.0 years, participated in the study. In addition, 49 healthy subjects (20 males; 29 females), aged 45.6±11.6 years, were recruited to serve as controls. Average Fatigue Severity Scale scores for the CFS group were 6.22±0.660, compared with scores of 3.04±1.23 for the healthy control group (p<0.0001). There were no significant differences in age or gender distribution between the groups.

Endogenous plasma L-carnitine, total carnitine, total acylcarnitine and individual acylcarnitine concentrations for the CFS patients and the healthy control groups are presented in Table 3. There were no significant differences in L-carnitine, total carnitine or total acylcarnitine levels between the groups. However, CFS patients had significantly lower concentrations of the C8:1, C14, C16:1, C18, C18:1 and C18:2 acylcarnitine concentrations than the healthy control subjects, with the mean acylcarnitine concentration in CFS patients being 74.2% (for C8:1), 81.5% (for C14), 80.5% (for C16:1), 84.9% (for C18), 69.6% (for C18:1) and 62.9% (for C18:2) of that of the healthy controls. Additionally, CFS patients had significantly higher C12DC and C18:1-OH acylcarnitine concentrations than the healthy control subjects, with the mean acylcarnitine concentration in normal patients being 72.4% (for C12DC) and 78.1% (for C18:1-OH) of that of the CFS patients. Of particular note was that the C18:1 and C18:2 acylcarnitines were markedly lower in CFS patients than healthy controls (FIG. 2; p<0.0001).

TABLE 3
Endogenous plasma carnitine concentrations (μmol/L)

| | Endogenous Plasma | | | |
|---|---|---|---|---|
| | Carnitine | Controls | CFS Patients | Significance |
| LC | L-carnitine | 45.2 ± 9.79 | 45.0 ± 11.3 | |
| TC | Total Carnitine | 59.5 ± 12.9 | 58.8 ± 13.6 | |
| AcylLC | Total Acylcarnitines | 14.3 ± 4.13 | 13.8 ± 3.45 | |
| C2 | Acetyl-L-carnitine | 10.6 ± 3.37 | 10.2 ± 2.72 | |
| C3 | Propionyl-L-carnitine | 0.502 ± 0.153 | 0.489 ± 0.199 | |
| C3DC | Malonyl-L-carnitine | 0.0447 ± 0.0217 | 0.0416 ± 0.0166 | |
| C4 | Butyryl-L-carnitine | 0.255 ± 0.0926 | 0.250 ± 0.107 | |
| C4—OH | Hydroxy-butyryl-L-carnitine | 0.0239 ± 0.0117 | 0.0252 ± 0.0130 | |
| C4DC | Succinyl-L-carnitine | 0.0750 ± 0.0135 | 0.104 ± 0.135 | |
| C5 | Isovaleryl-L-carnitine | 0.111 ± 0.0408 | 0.103 ± 0.0424 | |
| C5:1 | Tiglyl-L-carnitine | 0.0276 ± 0.00787 | 0.0285 ± 0.0110 | |
| C5—OH | Hydroxy-isovaleryl-L-carnitine | 0.0352 ± 0.00789 | 0.0392 ± 0.0103 | |
| C5DC | Glutaryl-L-carnitine | 0.129 ± 0.0419 | 0.132 ± 0.0575 | |
| C6 | Hexanoyl-L-carnitine | 0.0602 ± 0.0249 | 0.0605 ± 0.0213 | |
| C6:1 | Hexenoyl-L-carnitine | 0.0211 ± 0.00818 | 0.0222 ± 0.00988 | |
| C6DC | Adipyl-L-carnitine | 0.0947 ± 0.0121 | 0.0971 ± 0.0277 | |
| C8 | Octanoyl-L-carnitine | 0.0994 ± 0.0655 | 0.0953 ± 0.0553 | |
| C8:1 | Octenoyl-L-carnitine | 0.178 ± 0.117 | 0.132 ± 0.0752 * | p = 0.0201 |
| C8DC | Suberyl-L-carnitine | 0.0549 ± 0.00773 | 0.0601 ± 0.0177 | |
| C10 | Decanoyl-L-carnitine | 0.161 ± 0.113 | 0.154 ± 0.106 | |
| C10:1 | Decenoyl-L-carnitine | 0.105 ± 0.0520 | 0.109 ± 0.0536 | |
| C10:2 | Decadienoyl-L-carnitine | 0.0374 ± 0.0184 | 0.0395 ± 0.0216 | |
| C10DC | Sebacyl-L-carnitine | 0.0970 ± 0.0118 | 0.0999 ± 0.0162 | |
| C12 | Lauroyl-L-carnitine | 0.0668 ± 0.0364 | 0.0633 ± 0.0276 | |
| C12:1 | Dodecenoyl-L-carnitine | 0.0681 ± 0.0424 | 0.0707 ± 0.0437 | |
| C12DC | Dodecanedioyl-L-carnitine | 0.0782 ± 0.0124 | 0.108 ± 0.0489 * | p < 0.0001 |
| C14 | Myristoyl-L-carnitine | 0.0751 ± 0.0248 | 0.0612 ± 0.0269 * | p = 0.0023 |
| C14:1 | Myristoleyl-L-carnitine | 0.0694 ± 0.0435 | 0.0651 ± 0.0324 | |
| C14:2 | Tetradecadienoyl-L-carnitine | 0.0390 ± 0.0164 | 0.0398 ± 0.0177 | |
| C14—OH | Hydroxy-myristoyl-L-carnitine | 0.0149 ± 0.00667 | 0.0157 ± 0.00605 | |
| C16 | Palmitoyl-L-carnitine | 0.352 ± 0.148 | 0.404 ± 0.220 | |
| C16:1 | Palmitoleyl-L-carnitine | 0.0586 ± 0.0276 | 0.0472 ± 0.0212 * | p = 0.0383 |
| C16—OH | Hydroxyl-palmitoyl-L-carnitine | 0.0104 ± 0.00361 | 0.0114 ± 0.00570 | |
| C16:1-OH | Hydroxy-palmitoleyl-L-Carnitine | 0.0193 ± 0.00764 | 0.0211 ± 0.0111 | |
| C18 | Stearoyl-L-carnitine | 0.103 ± 0.0350 | 0.0874 ± 0.0326 * | p = 0.0104 |
| C18:1 | Oleyl-L-carnitine | 0.401 ± 0.170 | 0.279 ± 0.159 * | p < 0.0001 |
| C18:2 | Linoleyl-L-carnitine | 0.232 ± 0.111 | 0.146 ± 0.0911 * | p < 0.0001 |
| C18:1-OH | Hydroxy-oleyl-L-carnitine | 0.0178 ± 0.00858 | 0.0228 ± 0.0116 * | p = 0.0191 |

### Discussion

This study confirmed that CFS is not associated with alterations in plasma L-carnitine, total carnitine or total acylcarnitine levels; however, significant differences in the plasma concentration of particular individual acylcarnitines between CFS patients and healthy subjects were demonstrated for 8 of the 35 individual acylcarnitines quantified. The results clearly demonstrate a substantial reduction in oleyl-L-carnitine (C18:1) and linoleyl-L-carnitine (C18:2) concentrations in CFS patients. Additionally, significant reductions in the C8:1, C14, C16:1 and C18 acylcarnitine concentrations were observed, as well as a significant increase in the C12DC and C18:1-OH acylcarnitine concentration in CFS patients compared to healthy controls. Of the eight individual acylcarnitines found to have significant differences in CFS patients compared to healthy controls in the present study, Soetekouw et al. (2000) failed to find significant differences for five, and did not analyse the remaining three.

As long-chain fatty acids are the most energy-rich substrate for β-oxidation, small changes in acylcarnitine levels may have a significant impact on energy production, leading to fatigue. The deficiency in long-chain acylcarnitines observed in this study is indicative of a reduction in β-oxidation. Specifically, a lower plasma concentration of an acylcarnitine may indicate the transport of less long-chain acylcarnitines across the inner mitochondrial membrane, a corresponding reduction in the amount of acylcarnitines within the mitochondria that can undergo reverse transesterification by carnitine palmitoyltransferase II (CPT-II), and hence a reduction in long-chain fatty acid oxidation (as shown in FIG. 1). Accordingly, the results of the present study indicate that mitochondrial long-chain fatty acid β-oxidation is reduced in patients with CFS.

Based on the results of this study, it is anticipated that the administration of particular individual acylcarnitines, or alternatively, L-carnitine administered in combination with particular individual fatty acids (for example, oleic acid and linoleic acid) will be beneficial for the treatment of CFS. Supplementation with L-carnitine in combination with long-chain fatty acids may provide more substrate for long-chain acylcarnitine formation and/or concurrently increasing CPT-I activity. This, in turn, is expected to increase availability of long-chain acylcarnitines within the mitochondria and hence increase substrate availability for β-oxidation. Indeed, in a previous study (Maes et al., 2005) wherein endogenous levels of fatty acids were examined in 22 chronic fatigue syndrome patients and 12 healthy controls, it was demonstrated that CFS was accompanied by increased levels of omega-6 poly-unsaturated fatty acids and mono-unsaturated fatty acids. Interestingly, of the fatty acids measured in that study, for which the corresponding acylcarnitine was quantified in the present study (ie C14, C16, C16:1, C18, C18:1, C18:2), for five of the six cases there was a significant reduction in the acylcarnitine levels and an increase in the corresponding free fatty acid levels (ie C14, C16:1, C18, C18:1, C18:2). In fact, when the present findings are considered in combination with those of Maes et al., it can be speculated that the ratio of free fatty acid to acylcarnitine for these acyl groups is approximately 2- to 3-fold higher in CFS patients than in healthy controls, indicating a substantial disruption in fatty acid/carnitine homeostasis in these patients. While not wishing to be bound by theory, this may be due to either: (1) a reduction in the activity of AcylCoA synthase required for the conversion of free fatty acid to AcylCoA; or (2) a reduction in the activity of CPT-I. As CPT-I is the rate-controlling enzyme in mitochondrial fatty acid oxidation (Leonhardt et al., 2004), it is therefore anticipated that a reduction in CPT-I activity contributes to the symptomology of CFS.

In keeping with this, high levels of omega-6 fatty acids (such as C18:2 seen in the patient group of the present study) have been shown to inhibit CPT-I activity in rats (Niot et al., 1994), whereas an increase in the ratio of omega-3 to omega-6 fatty acids has been shown to increase CPT-I activity in both rats (Vamecq et al., 1993) and healthy controls (Beermann et al., 2003; Guebre-Egziabher et al., 2008). As L-carnitine is also known to increase CPT-I activity (Yoon et al., 2003), it is also anticipated that the administration of omega-3 fatty acids in combination with L-carnitine would stimulate CPT-I activity in CFS, thereby decreasing the ratio of free fatty acid to acylcarnitine and theoretically normalising mitochondrial fatty acid oxidation in these patients. Moreover, omega-3 fatty acids inhibit the production of malonyl-CoA, the major endogenous inhibitor of CPT-I, and reduce the sensitivity of CPT-I to inhibition by malonyl-CoA (Baker and Gibbons, 2000).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Amengual et al. (2008) Retinoic acid treatment increases lipid oxidation capacity in skeletal muscle of mice. Obesity 16(3):585-591.
Baker P W, Gibbons G F. (2000) Effect of dietary fish oil on the sensitivity of hepatic lipid metabolism to regulation by insulin. J Lipid Res 41(5):719-726.
Beermann C, Jelinek J, Reinecker T, Hauenschild A, Boehm G, Klor H U. (2003) Short term effects of dietary medium-chain fatty acids and n-3 long-chain polyunsaturated fatty acids on the fat metabolism of healthy volunteers. Lipids Health Dis 2(Art. 1):1-10.
Bieber L L. (1988) Carnitine. Annu Rev Biochem 57:261-283.
Bremer J. Carnitine—Metabolism and functions. (1983) Physiol Rev 63(4):1420-1480.
Brevetti G, Fanin M, De Amicis V, Carrozzo R, Di Lello F, Martone V D and Angelini C. (1997) Changes in skeletal muscle histology and metabolism in patients undergoing exercise deconditioning: effect of propionyl-L-carnitine. Muscle Nerve 20(9):1115-1120.
Chace D H, Hillman S L, Van Hove J L, Naylor E W. (1997) Rapid diagnosis of MCAD deficiency: Quantitative analysis of octanoylcarnitine and other acylcarnitines in newborn blood spots by tandem mass spectrometry. Clin Chem 43(11):2106-2113.
Chace D H, Kalas T A, Naylor E W. (2003) Use of tandem mass spectrometry for multianalyte screening of dried blood specimens from newborns. Clin Chem 49(11):1797-1817.
Guebre-Egziabher F, Rabasa-Lhoret R, Bonnet F, et al. (2008) Nutritional intervention to reduce the n-6/n-3 fatty acid ratio increases adiponectin concentration and fatty acid oxidation in healthy subjects. Eur Clin Nutr 62(11):1287-1293.
Jones M G, Goodwin C S, Amjad S, Chalmers R A. (2005) Plasma and urinary carnitine and acylcarnitines in chronic fatigue syndrome. Clin Chim Acta 360(1-2):173-177.
Krupp L B, LaRocca N G, Muir-Nash J, Steinberg A D. (1989) The fatigue severity scale. Application to patients with multiple sclerosis and systemic lupus erythematosus. Arch Neurol 46(10):1121-1123.
Kuratsune H, Yamaguti K, Takahashi M, Misaki H, Tagawa S, Kitani T. (1994) Acylcarnitine deficiency in chronic fatigue syndrome. Clin Infect Dis 18(Suppl 1):S62-S67.
Kuratsune H, Yamaguti K, Takahashi M, Tagawa S, Machii T, Kitani T. (1995) Abnormal Cellular Carnitine Metabolism in Chronic Fatigue Syndrome. EOS J Immunol Immunopharmacol 15(1-2):40-44.
Kuratsune H, Yamaguti K, Watanabe Y, et al. (1997) Acylcarnitine and Chronic Fatigue Syndrome. In: De Simone C, Famularo G, Editors. Carnitine Today. Austin, USA: RG Landes Company 195-213
Kuratsune H, Yamaguti K, Lindh G, et al. (1998) Low levels of serum acylcarnitine in chronic fatigue syndrome and chronic hepatitis type C, but not seen in other diseases. Int J Mol Med 2(1):51-56.
Leonhardt M, Langhans W. (2004) Fatty acid oxidation and control of food intake. Physiol Behav 83(4):645-651.
Maes M, Mihaylova I, Leunis J C. (2005) In chronic fatigue syndrome, the decreased levels of omega-3 poly-unsaturated fatty acids are related to lowered serum zinc and defects in T cell activation. Neuro Endocrinol Lett 26(6): 745-751.
Majeed T, De Simone C, Famularo G, Marcellini S, Behan P O. (1995) Abnormalities of carnitine metabolism in chronic fatigue syndrome. Eur J Neurol 2(5):425-428.
Malaguarnera M, Gargante M P, Cristaldi E, et al. (2008) Acetyl L-carnitine (ALC) treatment in elderly patients with fatigue. Archives of Gerontology and Geriatrics 46:181-190.
Niot I, Gresti J, Boichot J, et al. (1994) Effect of dietary n-3 and n-6 polyunsaturated fatty acids on lipid-metabolizing enzymes in obese rat liver. Lipids 29(7):481-489,
Plioplys A V, Plioplys S. (1995) Serum levels of carnitine in chronic fatigue syndrome: Clinical correlates. Neuropsychobiology 32(3):132-138.
Plioplys A V, Plioplys S. (1997) Amantadine and L-carnitine treatment of Chronic Fatigue Syndrome. Neuropsychobiology 35(1):16-23.
Pons R, De Vivo D C. (1995) Primary and secondary carnitine deficiency syndromes. J Child Neurol 10(Suppl 2):S8-S24.
Puri BK. (2004) The use of eicosapentaenoic acid in the treatment of chronic fatigue syndrome. Prostaglandins Leukot Essent Fatty Acids 70(4):399-401.
Puri BK. (2007) Long-chain polyunsaturated fatty acids and the pathophysiology of myalgic encephalomyelitis (chronic fatigue syndrome). J Clin Pathol 60(2):122-124.
Royal Australasian College of Physicians Working Group. (2002) Chronic fatigue syndrome clinical practice guidelines. MJA 176(9):S17-S56.
Scholte H R, Rodrigues Pereira R, de Jonge P C, Luyt-Houwen J E M, Verduin M H M, Ross J D. (1990) Primary carnitine deficiency. J Clin Chem Clin Biochem 28(5):351-357.
Soetekouw P M, Wevers R A, Vreken P, et al. (2000) Normal carnitine levels in patients with chronic fatigue syndrome. Neth J Med 57(1):20-24.
Tamizi far B, Tamizi B. (2002) Treatment of chronic fatigue syndrome by dietary supplementation with omega-3 fatty acids—A good idea? Med Hypotheses 58(3):249-250.
Taylor R R, Jason L A, Torres A. (2000) Fatigue rating scales: An empirical comparison. Psychol Med 30(4):849-856.
Vamecq J, Vallee L, de la Porte P L, et al. (1993) Effect of various n-3/n-6 fatty acid ratio contents of high fat diets on rat liver and heart peroxisomal and mitochondrial beta-oxidation. Biochim Biophys Acta 1170(2):151-156.
Vermeulen R C W, Scholte H R. (2004) Exploratory open label, randomized study of acetyl- and propionylcarnitine in chronic fatigue syndrome. Psychosom Med 66(2):276-282.
Yoon H R, Hong Y M, Boriack R L, Bennett M J. (2003) Effect of L-carnitine supplementation on cardiac carnitine palmitoyltransferase activities and plasma carnitine concentrations in adriamycin-treated rats. Pediatr Res 53(5):788-792.

1. A method of diagnosing chronic fatigue syndrome (CFS) in a test subject, said method comprising the steps of:
(i) determining a concentration of at least one individual acylcarnitine compound in a body sample from the test subject; and
(ii) comparing the concentration determined in step (i) to a reference concentration of the at least one individual acylcarnitine determined from an equivalent body sample from a healthy control subject (or a reference concentration range of the at least one individual acylcarnitine determined from equivalent body samples from a plurality of healthy control subjects),
wherein a difference in the concentration of the at least one individual acylcarnitine from the test subject compared to the reference concentration (or reference concentration range) is indicative of CFS in the test subject.

2. A method of diagnosing chronic fatigue syndrome (CFS) in a test subject, said method comprising the steps of:
(i) determining a concentration of at least one individual acylcarnitine compound in a first body sample from the test subject,
(ii) determining a concentration of at least one individual fatty acid that corresponds to an acyl group of said at least one individual acylcarnitine compound in a second body sample from the test subject, wherein said first and second body sample may be the same, and
(iii) determining a ratio of the concentration of the at least one individual acylcarnitine compound to the concentration of the at least one individual fatty acid, or
assessing a relationship between the concentration of the at least one individual acylcarnitine compound and the concentration of the at least one individual fatty acid;
wherein an aberrant ratio determined in step (iii) or an aberrant relationship assessed in step (iii) is indicative of CFS in the test subject.

3. A method of diagnosing chronic fatigue syndrome (CFS) in a test subject, said method comprising the steps of:
(i) determining a concentration of L-carnitine in a first body sample from the test subject, and
(ii) determining a concentration of at least one individual fatty acid that corresponds to an acyl group of at least one individual acylcarnitine compound in a second body sample from the test subject, wherein said first and second body sample may be the same, and
(iii) determining a ratio of the concentration of L-carnitine to the concentration of the at least one individual fatty acid, or
assessing a relationship between the concentration of the L-carnitine and the concentration of the at least one individual fatty acid;
wherein an aberrant ratio determined in step (iii) or an aberrant relationship assessed in step (iii) is indicative of CFS in the test subject.

4. The method of any one of claim 1, wherein the at least one individual acylcarnitine is a medium-chain or a long-chain acylcarnitine.

5. The method of claim 4, wherein the at least one individual acylcarnitine is selected from the group consisting of octenoyl-L-carnitine, dodecanedioyl-L-carnitine, myristoyl-L-carnitine, palmitoleyl-L-carnitine, stearoyl-L-carnitine, oleyl-L-carnitine, linoleyl-L-carnitine and hydroxyl-oleyl-L-carnitine.

6. The method of claim 5, wherein the at least one individual acylcarnitine is selected from oleyl-L-carnitine and linoleyl-L-carnitine.

7. The method of claim 1, wherein the body sample is plasma, serum or whole blood.

8. A method of treating chronic fatigue syndrome (CFS) in a subject, said method comprising administering an effective amount of a supplement comprising:
at least one acylcarnitine compound selected from short-chain, medium-chain and long-chain acylcarnitines,
L-carnitine, or an acylcarnitine that may be converted within a subject to L-carnitine, in combination with at least one fatty acid selected from short-chain, medium-chain and long-chain fatty acids, or
at least one acylcarnitine in combination with at least one fatty acid selected from short-chain, medium-chain and long-chain fatty acids.

9. The method of claim 8, wherein the supplement comprises at least one acylcarnitine selected from the group consisting of octenoyl-L-carnitine, dodecanedioyl-L-carnitine, myristoyl-L-carnitine, palmitoleyl-L-carnitine, stearoyl-L-carnitine, oleyl-L-carnitine, linoleyl-L-carnitine and hydroxyl-oleyl-L-carnitine.

10. The method of claim 9, wherein the supplement comprises oleyl-L-carnitine or linoleyl-L-carnitine.

11. The method of claim 8, wherein the supplement comprises two or more individual acylcarnitine compounds wherein at least one of the individual acylcarnitines is selected from medium-chain and long-chain acylcarnitines, or the supplement comprises L-carnitine, or an acylcarnitine that may be converted within a subject to L-carnitine, in combination with two or more individual fatty acids wherein at least one of the individual fatty acids is selected from medium-chain and long-chain fatty acids.

12. The method of claim 11, wherein the supplement comprises oleyl-L-carnitine and linoleyl-L-carnitine.

13. The method of claim 8, wherein the supplement comprises at least one individual fatty acid selected from the group consisting of octenoic acid, dodecanedioic acid, myristoic acid, palmitoleic acid, stearoic acid, oleic acid, linoleic acid and hydroxyl-oleic acid.

14. The method of claim 13, wherein the supplement comprises oleic acid and/or linoleic acid.

15. The method of claim 8, wherein the method further comprises administering a modulator(s) of any one or more of carnitine palmitoyltransferase (CPT)-I, carnitine palmitoyltransferase (CPT)-II and carnitine/acylcarnitine translocase.

16. The method of claim 15, wherein the modulator(s) is selected from omega-3 fatty acids.

17. The method of claim 16, wherein the modulator(s) is eicospentaenoic acid (EPA) and/or docosahexaenoic acid (DHA).

18. The method of claim 15, wherein the modulator(s) is provided in the said supplement.

19. A method of treating chronic fatigue syndrome (CFS) in a subject, said method comprising the steps of:
(i) identifying in said subject a deficiency in one or more individual acylcarnitine compound(s) by
(a) determining a concentration of at least one individual acylcarnitine in a test body sample from the subject, and
(b) comparing the concentration determined in (a) to a reference concentration of the at least one individual acylcarnitine determined from an equivalent body sample(s) from a healthy control subject (or a reference concentration range of the at least one individual acylcarnitine determined from equivalent body samples from a plurality of healthy control subjects), wherein a lesser concentration of the at least one individual acylcarnitine compound(s) from the subject compared to the reference concentration (or reference concentration range) indicates a deficiency in the said at least one individual acylcarnitine compound(s); and
(ii) administering an effective amount of a supplement comprising the deficient at least one individual acylcarnitine compound(s), L-carnitine (or an acylcarnitine that may be converted within a subject to L-carnitine) in combination with at least one fatty acid that corresponds to the deficient at least one individual acylcarnitine compound(s), or the deficient at least one individual acylcarnitine compound(s) in combination with at least one fatty acid that corresponds to the deficient at least one individual acylcarnitine compound(s).

20. The method of claim 19, wherein the supplement comprises at least one acylcarnitine selected from the group consisting of octenoyl-L-carnitine, dodecanedioyl-L-carnitine, myristoyl-L-carnitine, palmitoleyl-L-carnitine, stearoyl-L-carnitine, oleyl-L-carnitine, linoleyl-L-carnitine and hydroxyl-oleyl-L-carnitine.

21. The method of claim 20, wherein the supplement comprises oleyl-L-carnitine or linoleyl-L-carnitine.

22. The method of claim 19, wherein the supplement comprises L-carnitine (or an acylcarnitine that may be converted within a subject to L-carnitine) in combination with at least one fatty acid selected from the group consisting of octenoic acid, dodecanedioic acid, myristoic acid, palmitoleic acid, stearoic acid, oleic acid, linoleic acid and hydroxyl-oleic acid.

23. The method of claim 22, wherein the supplement comprises L-carnitine (or an acylcarnitine that may be converted within a subject to L-carnitine) in combination with oleic acid and/or linoleic acid.

24. The method of claim 19, wherein the method further comprises administering a modulator(s) of any one or more of carnitine palmitoyltransferase (CPT)-I, carnitine palmitoyltransferase (CPT)-II and carnitine/acylcarnitine translocase.

25. The method of claim 24, wherein the modulator(s) is selected from omega-3 fatty acids.

26. The method of claim 25, wherein the modulator(s) is eicospentaenoic acid (EPA) and/or docosahexaenoic acid (DHA).

27. The method of claim 24, wherein the modulator(s) is provided in the said supplement.

28. The method of claim 19, wherein the body sample is plasma, serum or whole blood.

29. A method of fortifying a food comprising adding to the food a supplement comprising;
at least one acylcarnitine compound selected from short-chain, medium-chain and long-chain acylcarnitines,
L-carnitine, or an acylcarnitine that may be converted within a subject to L-carnitine, in combination with at least one fatty acid selected from short-chain, medium-chain and long-chain fatty acids, or
at least one acylcarnitine in combination with at least one fatty acid selected from short-chain, medium-chain and long-chain fatty acids.

30. The method of claim 29, wherein the supplement comprises at least one acylcarnitine selected from the group consisting of octenoyl-L-carnitine, dodecanedioyl-L-carnitine, myristoyl-L-carnitine, palmitoleyl-L-carnitine, stearoyl-L-carnitine, oleyl-L-carnitine, linoleyl-L-carnitine and hydroxyl-oleyl-L-carnitine.

31. The method of claim 30, wherein the supplement comprises oleyl-L-carnitine or linoleyl-L-carnitine.

32. The method of claim 29, wherein the supplement comprises L-carnitine (or an acylcarnitine that may be converted within a subject to L-carnitine) in combination with at least one fatty acid selected from the group consisting of octenoic acid, dodecanedioic acid, myristoic acid, palmitoleic acid, stearoic acid, oleic acid, linoleic acid and hydroxyl-oleic acid.

33. The method of claim 32, wherein the supplement comprises L-carnitine (or an acylcarnitine that may be converted within a subject to L-carnitine) in combination with oleic acid and/or linoleic acid.

34. The method of claim 29, wherein the method further comprises administering a modulator(s) of any one or more of carnitine palmitoyltransferase (CPT)-I, carnitine palmitoyltransferase (CPT)-II and carnitine/acylcarnitine translocase.

35. The method of claim 34, wherein the modulator(s) is selected from omega-3 fatty acids.

36. The method of claim 35, wherein the modulator(s) is eicospentaenoic acid (EPA) and/or docosahexaenoic acid (DHA).

37. The method of claim 34, wherein the modulator(s) is provided in the said supplement.

38. A method of treating chronic fatigue syndrome (CFS) in a subject, said method comprising administering an effective amount of a modulator(s) of carnitine/acylcarnitine metabolism wherein the modulator stimulates the activity of an enzyme selected from the group consisting of carnitine palmitoyltransferase (CPT)-I, carnitine palmitoyltransferase (CPT)-II and carnitine/acylcarnitine translocase.

39. The method of claim 38, wherein the modulator(s) is selected from the group consisting of L-carnitine (or an acylcarnitine that may be converted within a subject to L-carnitine), all-trans retinoic acid, fatty acids and combinations thereof.

40. The method of claim 39, wherein the modulator(s) comprises L-carnitine (or an acylcarnitine that may be converted within a subject to L-carnitine) in combination with one or more omega-3 fatty acids.

41. The method of claim 40, wherein the modulator(s) comprises L-carnitine (or an acylcarnitine that may be converted within a subject to L-carnitine) in combination with eicospentaenoic acid (EPA) and/or docosahexaenoic acid (DHA).
